(19) United States
(12) Patent Application Publication
(10) Pub. No.: US 2023/0072820 A1
(43) Pub. Date: Mar. 9, 2023

(54) VACCINE SITE ASSESSMENT SIMULATOR

(71) Applicant: Rowan University, Glassboro, NJ (US)

(72) Inventors: Ali A. Houshmand, Mullica Hill, NJ (US); George D. Lecakes, JR., Laurel Springs, NJ (US)

(21) Appl. No.: 17/898,002

(22) Filed: Aug. 29, 2022

Related U.S. Application Data
(60) Provisional application No. 63/238,896, filed on Aug. 31, 2021.

(51) Int. Cl. G16H 20/17; G16H 40/20

(52) U.S. Cl. CPC G16H 20/17; G16H 40/20

(57) ABSTRACT

A computer product, including a logical environment system adapted to generate a logical network comprising a plurality of logically related, rule based junctions through which one or more entities traverse, a spatial environment system adapted to map one or more spatial constraints upon each logical relation between each of the one or more of the plurality of junction; and a rendering environment system adapted to move the one or more entities through the logical network subject to the corresponding spatial constraints and to visually display the results of the movement of the one or more entities via a graphical user interface.

### CROSS-REFERENCE TO RELATED APPLICATION

This application claims the benefit of priority of U.S. Provisional Patent Application No. 63/238,896, filed Aug. 31, 2021, the entire disclosure of which is hereby incorporated herein by reference.

### FIELD OF THE INVENTION

The present invention relates generally to controlling a user interface of a computerized system to provide a simulated virtual-reality type information display and user experience, and more particularly to providing a display of information on a computerized system that simulates human navigation in a rule-bounded environment.

### BACKGROUND

It is known to create bespoke models of physical systems. Such models typically rely solely on archival data as inputs and operate to produce a data set indicative of the state of the model at some defined time or in some defined state. Such models are often times substantially customized to represent a particular environment and, as such, to not provide utility outside of fairly narrow use scenarios.

What is therefore needed is a computerized system and method for simulating a physical system that is easily customizable and applicable to a broad range of use scenarios and is capable of real-time data augmentation.

### SUMMARY

The present invention provides a computerized system and method for simulating a physical system that is easily customizable and applicable to a broad range of use scenarios and is capable of real-time data augmentation. In accordance with exemplary and non-limiting embodiments, a computer product comprises a logical environment system adapted to generate a logical network comprising a plurality of logically related, rule based junctions through which one or more entities traverse, a spatial environment system adapted to map one or more spatial constraints upon each logical relation between each of the one or more of the plurality of junctions and a rendering environment system adapted to move the one or more entities through the logical network subject to the corresponding spatial constraints and to visually display the results of the movement of the one or more entities via a graphical user interface.

In accordance with exemplary and non-limiting embodiments, a computer-implemented method for controlling a user interface comprises generating a logical network comprising a plurality of logically related, rule-based junctions through which one or more entities traverse, mapping one or more spatial constraints upon each logical relation between each of the one or more of the plurality of junctions and moving the one or more entities through the logical network subject to the corresponding spatial constraints and visually displaying the results of the movement of the one or more entities via a graphical user interface on a display system.

### BRIEF DESCRIPTION OF THE FIGURES

For a better understanding of the present invention, reference may be made to the accompanying drawings in which:

FIG. 1 is a system diagram showing an exemplary network computing environment in which the present invention may be employed;

FIG. 2 illustrates an exemplary and non-limiting embodiment of a computerized system in accordance with the present invention;

FIGS. 3A-3D illustrate an exemplary and non-limiting embodiment of a logical node topology;

FIG. 4 illustrates an exemplary and non-limiting embodiment of a simulation setup GUI element;

FIG. 5 illustrates an exemplary and non-limiting embodiment of a perspective view of a simulated environment;

FIG. 6 illustrates an exemplary and non-limiting embodiment of a flowchart; and

FIGS. 7A-7D illustrate an exemplary and non-limiting embodiment of a flowchart.

### DETAILED DESCRIPTION

The present invention provides a computerized system and method for simulating a physical system that is easily customizable and applicable to a broad range of use scenarios and is capable of real-time data augmentation. In accordance with exemplary and non-limiting embodiments, a computer product comprises a logical environment system adapted to generate a logical network comprising a plurality of logically related, rule based junctions through which one or more entities traverse, a spatial environment system adapted to map one or more spatial constraints upon each logical relation between each of the one or more of the plurality of junctions and a rendering environment system adapted to move the one or more entities through the logical network subject to the corresponding spatial constraints and to visually display the results of the movement of the one or more entities via a graphical user interface.

This functionality is implemented by a computerized system and/or computing system specially-configured in accordance with the present invention. The functionality of the system may be implemented by a local computing system alone, or by a local computing system that is in communication with a remote computer system, e.g., via a communications network such as the internet.

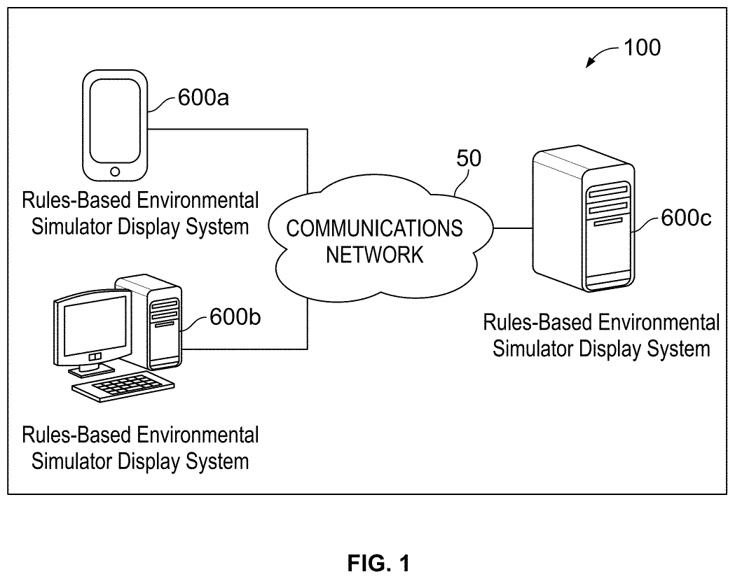

According to illustrative embodiment(s) of the present invention, various views are illustrated in FIGS. 1-7D and like reference numerals are used consistently throughout to refer to like and corresponding parts of the invention for all of the various views and figures of the drawings.

The following detailed description of the invention contains many specifics for the purpose of illustration. Any one of ordinary skill in the art will appreciate that many variations and alterations to the following details are within scope of the invention. Accordingly, the following implementations of the invention are set forth without any loss of generality to, and without imposing limitations upon, the claimed invention.

System Environment

An exemplary embodiment of the present invention is discussed below for illustrative purposes. FIG. 1 is a system diagram showing an exemplary network computing environment 100 in which the present invention may be employed. As shown in FIG. 1, the exemplary network environment 100 includes conventional computing hardware and software for communicating via a communications network 50, such as the Internet, etc., using a Rules-Based Environmental Simulator Display System (RBESDS) 600a, 600b, each of which may be, for example, one or more personal computers/PCs, laptop computers, tablet computers, smartphones, or other computing system hardware, including computerized/networked communication hardware/software/functionality, such as computer-based servers, kiosks and the like.

In accordance with a certain aspect of the present invention, one or more of the Rules-Based Environmental Simulator Display Systems (RBESDSs) 600a, 600b may store and execute an “app” or other purpose-specific application software in accordance with the present invention, although this is not required in all embodiments. In other embodiments, a SaaS or internet/web-based server and/or software platform may be used to deliver similar functionality to the RBESDSs 600a, 600b. Hardware and software for enabling communication of data by such systems via such communications networks are well known in the art and beyond the scope of the present invention, and thus are not discussed in detail herein.

### Rules-Based Environmental Simulator Display System

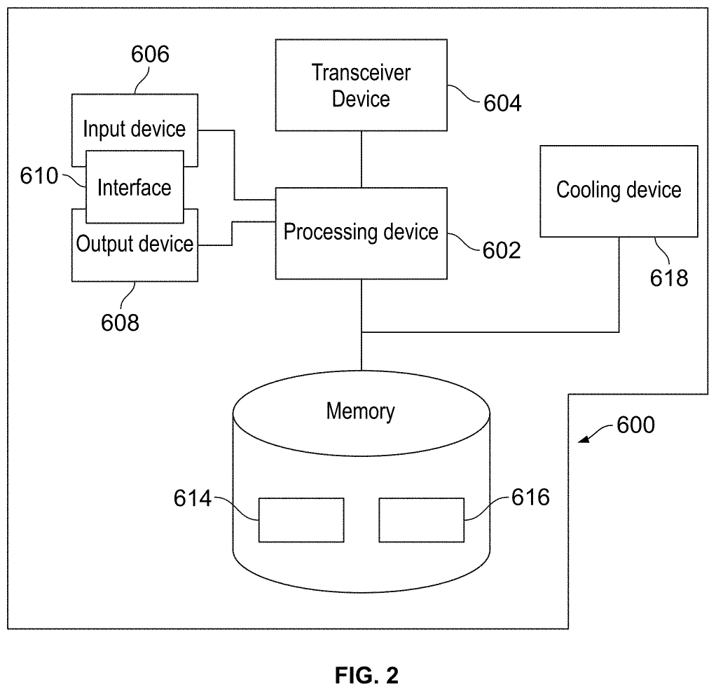

FIG. 2 is a schematic block diagram showing an exemplary Rules-Based Environmental Simulator Display System (RBESDS) 600 in accordance with an exemplary embodiment of the present invention. The RBESDS 600 is a special-purpose computer system that includes conventional computing hardware storing and executing both conventional software enabling operation of a general purpose computing system, such as operating system software, network communications software, and specially-configured computer software for configuring the general purpose hardware as a special-purpose computer system for carrying out at least one method in accordance with the present invention. By way of example, the communications software may include conventional web server software, and the operating system software may include iOS, Android, Windows, Linux software.

Accordingly, the exemplary RBESDS 600 of FIG. 2 includes a general-purpose processor, such as a microprocessor (CPU) or other processing system 602 and a bus employed to connect and enable communication between the processor 602 and the components of the system in accordance with known techniques. The exemplary system 600 includes a user interface 610, which connects the processor 602 via the bus to one or more input systems 606, such as a keyboard, mouse, a camera and/or other interface systems, which can be any user interface system, such as a camera, microphone, touch sensitive screen, digitized entry pad, etc. The bus also connects an output system 608, such as an LCD screen or monitor, to the processor 602, e.g., via a display adapter. The bus also connects the processor 602 to memory 218, which can include a hard drive, diskette drive, tape drive, etc.

The RBESDS 600 may communicate with other computers or networks of computers, for example via a communications channel, network card or modem. The RBESDS 600 may be associated with such other computers in a local area network (LAN) or a wide area network (WAN), and may operate as a server in a client/server arrangement with another computer, etc. Such configurations, as well as the appropriate communications hardware and software, are known in the art.

The RBESDS 600 may be a specially-configured in accordance with the present invention. Accordingly, as shown in FIG. 2, the RBESDS 600 includes computer-readable, processor-executable instructions stored in the memory for carrying out the methods described herein. Further, the memory stores certain data, e.g., in one or more databases or other data stores shown in FIG. 2 for illustrative purposes, without regard to any particular embodiment in one or more hardware or software components.

Referring again to FIG. 2, there is illustrated a block diagram of an RBESDS computer system 600 according to some embodiments is shown. In some embodiments, the RBESDS 600 may, for example, execute, process, facilitate, and/or otherwise be associated with the embodiments described above. In some embodiments, the system 600 may comprise a processing system 602, a transceiver system 604, an input system 606, an output system 608, an interface 610, a memory system 612 (storing various programs and/or instructions 614 and data 616), and/or a cooling system 618. According to some embodiments, any or all of the components 602, 604, 606, 608, 610, 612, 614, 616, 618 of the system 600 may be similar in configuration and/or functionality to any similarly named and/or numbered components described herein. Fewer or more components 602, 604, 606, 608, 610, 612, 614, 616, 618 and/or various configurations of the components 602, 604, 606, 608, 610, 612, 614, 616, 618 be included in the apparatus 600 without deviating from the scope of embodiments described herein.

According to some embodiments, the processor 602 may be or include any type, quantity, and/or configuration of processor that is or becomes known. In some embodiments, the processor 602 may comprise multiple inter-connected processors, microprocessors, and/or micro-engines. According to some embodiments, the processor 602 (and/or the system 600 and/or other components thereof) may be supplied power via a power supply (not shown), such as a battery, an Alternating Current (AC) source, a Direct Current (DC) source, an AC/DC adapter, solar cells, and/or an inertial generator. In the case that the system 600 comprises a server, such as a blade server, necessary power may be supplied via a standard AC outlet, power strip, surge protector, and/or Uninterruptible Power Supply (UPS) system.

In some embodiments, the transceiver system 604 may comprise any type or configuration of communication system that is or becomes known or practicable. The transceiver system 604 may, for example, comprise a Network Interface Card (NIC), a telephonic system, a cellular network system, a router, a hub, a modem, and/or a communications port or cable. According to some embodiments, the transceiver system 604 may also or alternatively be coupled to the processor 602. In some embodiments, the transceiver system 604 may comprise an IR, RF, Bluetooth™′ Near-Field Communication (NFC), and/or Wi-Fi® network system coupled to facilitate communications between the processor 602 and another system (not shown).

According to some embodiments, the input system 606 and/or the output system 608 may be communicatively coupled to the processor 602 (e.g., via wired and/or wireless connections and/or pathways) and they may generally comprise any types or configurations of input and output components and/or systems that are or become known, respectively. The input system 606 may comprise, for example, a keyboard that allows an operator of the system 600 to interface with the system 600. The output system 606 may, according to some embodiments, comprise a display screen and/or other practicable output component and/or system. According to some embodiments, the input system 608 and/or the output system 606 may comprise and/or be embodied in a single system, such as a touch-screen monitor or display.

The memory system 612 may comprise any appropriate information storage system that is or becomes known or available, including, but not limited to, units and/or combinations of magnetic storage systems (e.g., a hard disk drive), optical storage systems, and/or semiconductor memory systems, such as RAM systems, Read Only Memory (ROM) systems, Single Data Rate Random Access Memory (SDR-RAM), Double Data Rate Random Access Memory (DDR-RAM), and/or Programmable Read Only Memory (PROM). The memory system 612 may, according to some embodiments, store one or more software components.

According to some embodiments, the simulation software 614 may be operable to cause the processor 612 to process entity data, such as from entity database 616. Any or all of the exemplary instructions and data types described herein and other practicable types of data may be stored in any number, type, and/or configuration of memory systems that is or becomes known. The memory system 612 may, for example, comprise one or more data tables or files, databases, table spaces, registers, and/or other storage structures. In some embodiments, multiple databases and/or storage structures (and/or multiple memory systems 612) may be utilized to store information associated with the system 600. According to some embodiments, the memory system 612 may be incorporated into and/or otherwise coupled to the system 600 (e.g., as shown) or may simply be accessible to the system 600 (e.g., externally located and/or situated).

System Operation

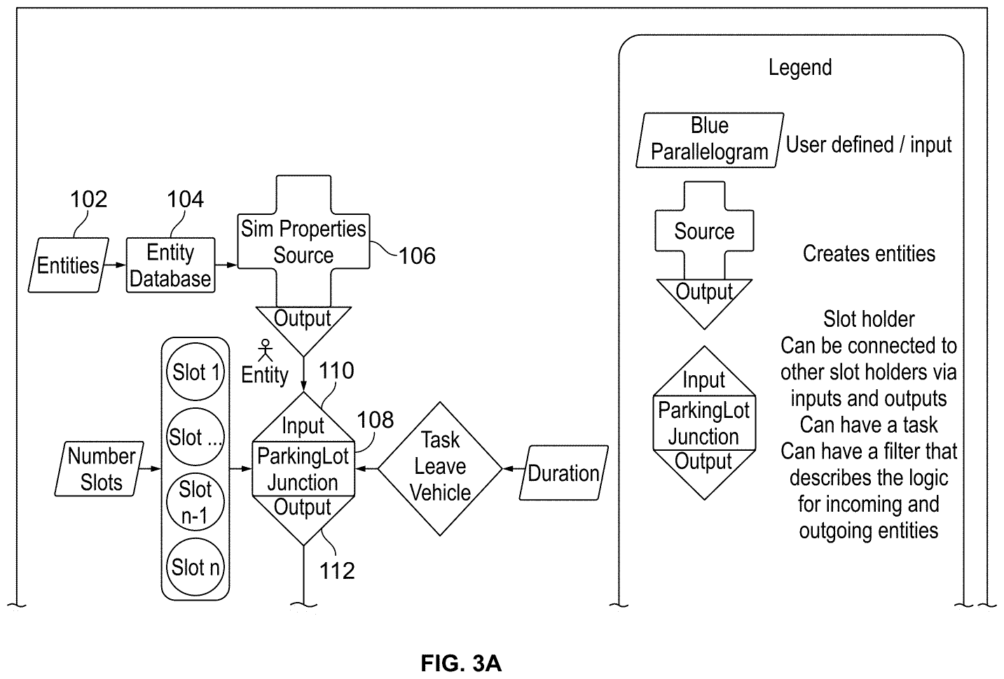

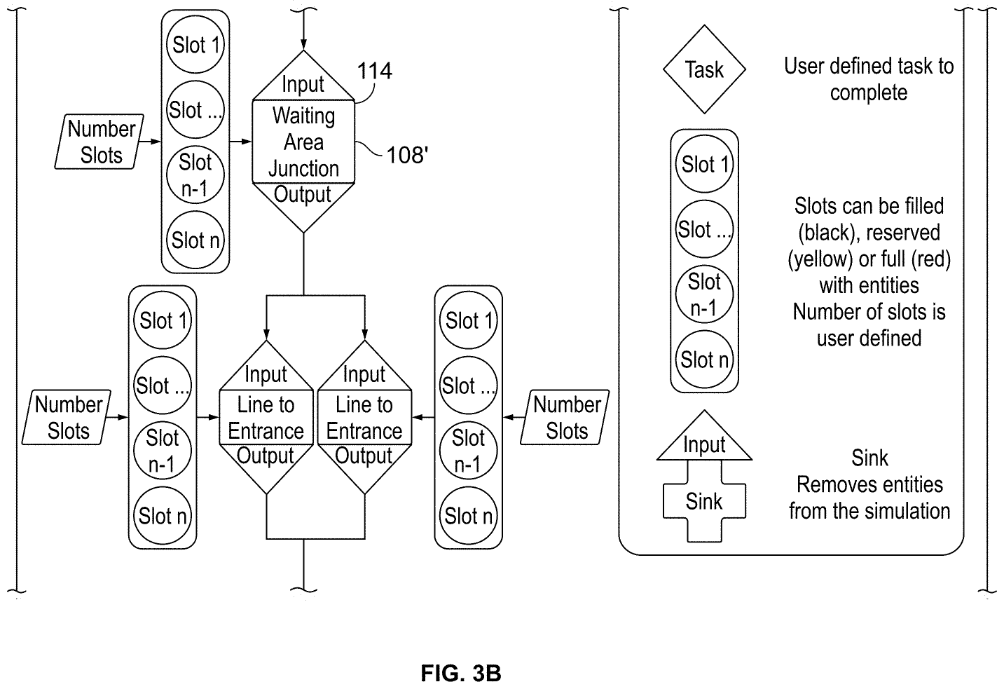

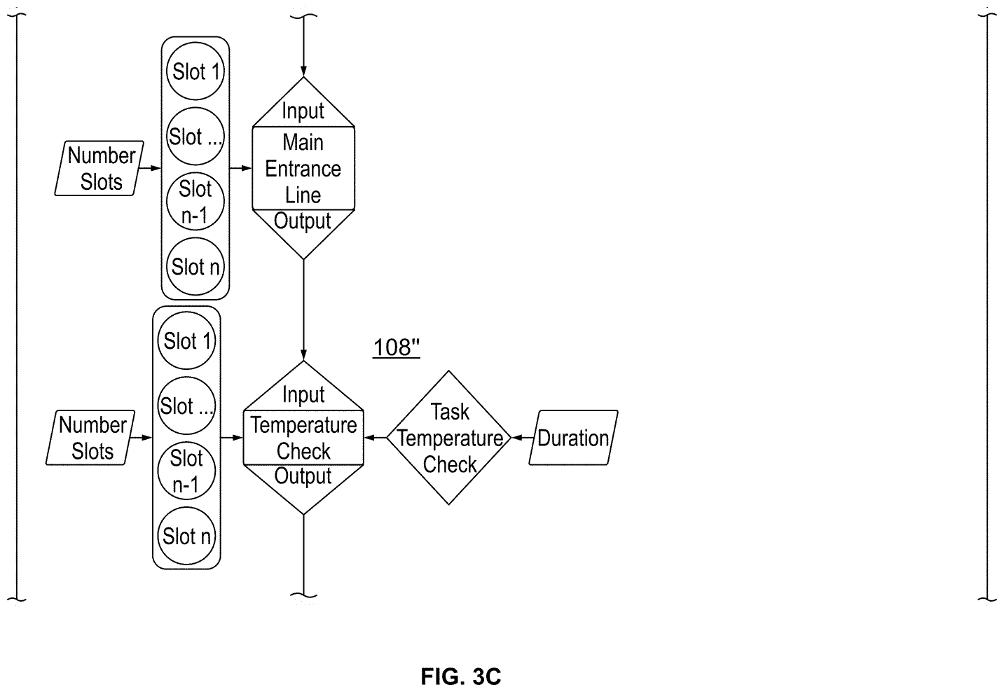

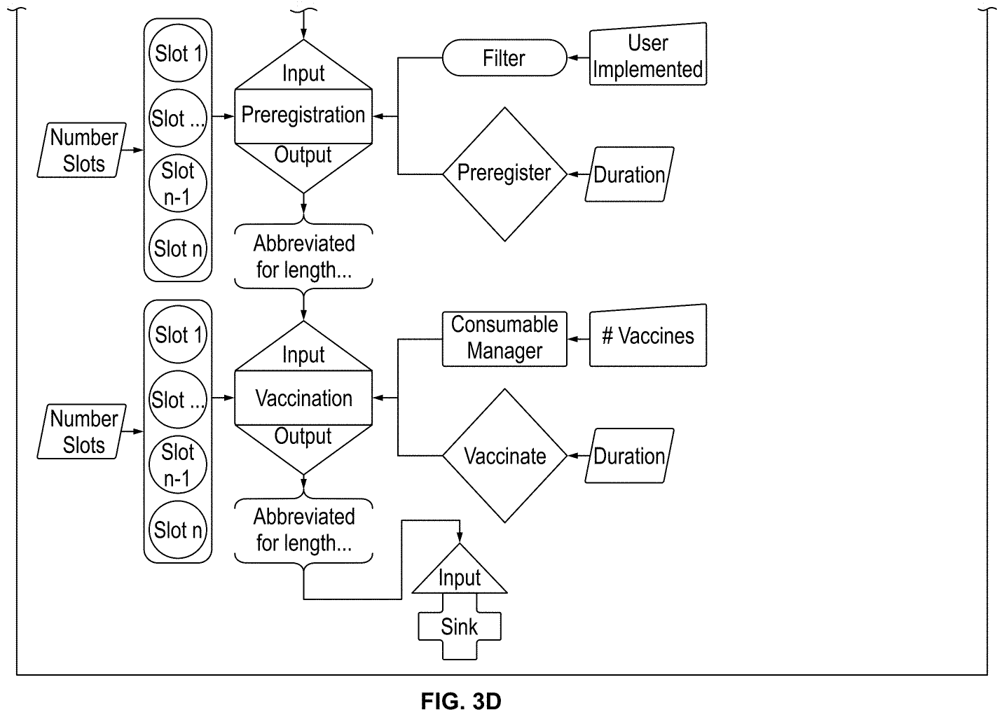

Exemplary operation of the RBESDS 600 system of FIGS. 1 and 2 is discussed below with reference to FIGS. 3A-7D.

In accordance with exemplary and non-limiting embodiments, an exemplary process enables a user to create, interact and modify a series of extendable and modular processes for use in simulating real-life tasks, allowing data to flow between each process to simulate a real-life process utilizing spatial relationships. Data input may be set up by the user and may enter the pipeline where it may be processed by a series of modular and extendable stages. Sources and sinks may control data inflow and outflow from the system, where data may move from stage to stage, and be subject to user-dictated outcomes.

Each element may process data in series or parallel, keeping track of important parameters such as time to complete and current progress through the system. The user may define a series of conditional statements that define how data flows into the system, how data is handled at each stage, and how it is removed. Attributes of each data element may be tracked and recorded to provide progress. A data element may be composed of several attributes, the values of which may be directly set by the user or inferred through probability controls set by the user at the start of the simulation. After creation, the properties of the stage, such as duration and number of data objects that can be processed in parallel may be modified.

In accordance with exemplary and non-limiting embodiments, the RBESDS 600 provides a software-based simulation platform for moving user defined data entities through a connected node topology pipeline whereby each node performs predefined functionality upon each entity in accordance with the logical attributes of the entity and the spatial attributes of an environment.

The platform allows for a purely logical definition of the paths or paths through which one or more entities may travers. Nodes along these paths may have accompanying rules that determine when and under what conditions and entity may access or arrive at the node as well as what conditions may permit the entity to move to another node. In some instances, as discussed more fully below, there may exist occupancy rules defining how many entities may reside at a node. As a result, the ability of an entity to exit from one entity towards another may depend, at least in part, on there being room for the entity at the node to which the entity intends to travel.

For example, a node in a logical network detailing the flow of people through a vaccine site may have a node representing a check-in desk with the next node after check-in representing a waiting room. If the waiting room node is filled to capacity, an entity may need to exit the waiting room node before an entity may be released from the check-in node to travel logically to the waiting room node.

In the broadest sense, entities may be any data structure describing a physical class of objects. For example, an entity may be a person requiring a vaccination. The data structure defining each individual person may comprise any number of descriptive attributes of each person. A spatial system translates the logical node topology to a simulated physical environment having real world proportions through which an entity may be moved in a simulated manner. While decoupled one from the other, the logical node topology and the simulated physical environment function together to realistically simulate an environment in which entities move through a simulated physical environment.

In some exemplary embodiments, the movement of entities through the logical node topology in accordance with the dictates and limitations of the defined spatial system may be visually presented to a user of the system. In some instances, this visualization may serve as a way for a system administrator to visualize, query and otherwise interact with a modeled scenario. In other instances, individuals associated with one or more of the entities may be provided a visualization from the point of view of one of the entities. For example, an individual experiencing anxiety over the prospect of parking and finding her way to the vaccine facility may be provided with a visualization of moving through the modeled environment. The visualization may be derived from a real-time or near real-time execution of a system simulation.

In accordance with exemplary and non-limiting embodiments, the connected node topology is a logical description of a physical environment. For example, a vaccination site may be logically represented by nodes (e.g., parking lot junction, temperature check, etc.) which are logically connected with regards to which node outputs form which node inputs (e.g., entities proceed from “main entrance line” node (output) to “temperature check” node (input)).

As noted and described more fully below, in accordance with exemplary and non-limiting embodiments, the connected node topology is a logical description of a physical environment. For example, a vaccination site may be logically represented by nodes (e.g., parking lot junction, temperature check, etc.) which are logically connected with regards to which node outputs form which node inputs (e.g., entities proceed from “main entrance line” node (output) to “temperature check” node (input)).

With reference to FIGS. 3A-3D, there is illustrated an exemplary and non-limiting embodiment of a logical node topology. In the illustrated example, the system is directed to moving entities comprising individuals through a vaccine site. Logical node topology 100 is comprised of a plurality of slot holders 108 each of which represents a logical processing unit that operates to move entities through the simulation. Each slot holder 114 is comprised of a junction 114 associated with a task to be completed. Each task is an operation that must be completed before moving to the next slot holder 108. Slot holders 108 are related to one another via their inputs and outputs. Specifically, when an entity is outputted by a slot holder, the entity forms the input for another slot holder 108.

For example, an entity comprising an individual may be output from a simulation properties source 106. This outputted entity may form the input slot holder 108 “Parking Lot Junction.” As illustrated, slot holder 108 has an associated task with a defined duration for the task. In this instance, upon arriving at the parking lot junction, an entity is required to perform the task of leaving a vehicle. As a result, upon the passage of a user definable duration of time required to leave a vehicle, the entity may be outputted so as to form an input to the waiting area junction slot holder 108′. Each slot holder 108 may be comprised of an associated user definable number of slots 116. As illustrated, individual slots may have variable statuses that indicate the availability of slots. In some instances, an entity that is prepared to be outputted may not form the input to a subsequent slot holder 108 in the absence of an available slot 116 at the subsequent slot holder 108.

In this manner, entities progress from one slot holder 108 to the next. As illustrated, entities 102 are fed into an entity database 104 forming the input into the simulation properties source 106.

As described, the logical, node-based definition of the system is linked to a corresponding physically defined spatial model. For example, a parking lot node may be logically linked to an in-processing desk inside of a hospital. As a result, entities that logically reside at the parking lot node will move to the in-processing desk in accordance with the rules and availability attribute values present at each node. Once it is determined that an entity is to move from the parking lot to the in-processing desk, updating of the occupancy attribute of the in-processing desk node may pause until the entity has successfully traversed from the parking lot to the in-processing desk in the corresponding physical model. In some instances, there may be communication between the physical model simulation and the logical representation of the system to alert the system that an entity is “lost” or unable to successfully traverse the defined system.

For example, the physical model of the parking lot and the path to the in-processing desk may contain defined barriers or attributes which deter or delay an entity from travelling from the parking lot to the in-processing desk. In some embodiments, entities obeying the limitations and strictures of the physical model may find themselves effectively “lost” and unable to proceed within the physical model in a manner dictated by the logical definition of the system. In such instances,

As implemented in computer code, slot holders 108 and junctions 114 may be embodied as classes to which may be added custom functionality. In addition, lines may form an additional class. Slot holders 108, junctions 114 and lines may operate on entity objects, which, in the example above, represent patients. Entities may be processed by slot holders as they move through the simulation. Slot holders may be connected to one another as inputs and outputs. Junctions represent areas to complete a task, which is an operation an entity must complete before moving on to the next Slot Holder. Junctions appear to operate in parallel, with each entity working independent of the others. Lines represent a list of entities moving through space starting at the end and working their way to the start. Entities are processed in order and can only move forward when there is an open Slot. A slot is a base class that represents an open area. Lines may generate slots using paths and the social distancing parameter set at the start of the simulation. For junctions, slots much be manually placed, or an algorithm implemented to automatically place them in the simulation space. In the next few sections, a more detailed explanation of the classes is provided as well as examples on how to extend them to work with a unique user defined site.

The Slot holder class is the base class for both junctions 114 and lines, which make up nearly all connections for routing entities within the simulation. The slot holder class contains several abstract methods that can be implemented to create your own way of handling the movement of entities. Such methods are as follows:

abstract protected void CheckNextSetOfSlots( ); Evaluates the next batch of slots held by the slot holder. This portion of code should handle how an entity moves through the slot holder and call the appropriate methods such as NoOutput( ), OutputFull( ), or OutputOpen( ). This method is usually the first to be implemented when a new method of routing entities needs to be created.

abstract protected void GenerateSlots( ); Handles creating the slots from the slot spawn points and creates and visual representation in the 3D world.

abstract public bool AddEntity(Entity entity, bool fromSource=false); Called when an entity is being added to the slot holder. Includes a parameter indicating if the entity is being generated from a source or if it is coming from another slot holder. If the entity is generated from a source it is recommended to instantaneously move the entity rather than have it path find to the location since a source may not have a physical location. A Boolean value is returned to indicate if the entity was successfully added, however it is recommended to first test the slot holder using Is Full( ) to determine if it is available to handle additional entities.

abstract protected void NoOutput(Slot slot); Called when there is no output connected and an entity has reached the end of the slot holder and is trying to move to the next location.

abstract protected void OutputFull(Slot slot); Called when the output location is full that the entity is trying to move to.

abstract public void OutputOpen(SlotHolder output, Slot slot); Called when the output location can accept an entity.

abstract public bool IsFull( ); Should include the code necessary to determine if the slot holder is full.

Junctions represent a location that features a task to be completed in some length of time where each entity can work in parallel. A junction iterates over a set number of entities based on the performance of the system being used and checks if they are ready to move to the next location. The next location is queried and based on the results one of the following methods is called: NoOutput, OuputFull, OutputOpen.

When extending a Junction, the most common method to override is the OutputOpen(SlotHolder output, Slot slot). In the below Example 1, a custom junction was created to handle entity registration. In this situation, a simulation property Boolean is set to true to track the progress of the entity through the simulation when it has completed the task and is moving to a new Slot Holder.

### Example 1

public class Registration : Junction
{
public override void OutputOpen(SlotHolder output, Slot slot)
{
Suppertimes properties =
slot.GetEntity( ).GetComponent<SimProperties>( );
if (properties)
{
properties.IsRegistered = true;
}
base.OutputOpen(output, slot);
}
}

Lines represent an ordered queue of entities that move from the last slot to the first in order. When an entity reaches the start of a line but there is no connected output the NoOutput( ) method is called. In the below Example 2, a line is extended to handle the condition where the entity has completed an entire pass through the simulation and must find their vehicle where they spawned from. In this case, because the entity is meant to leave the simulation, it does not matter whether the output exists, is available, or full. Either way, the entity is told to return to their origin.

### Example 2

public class LineToOrigin : Line {
protected override void NoOutput(Slot slot){
Entity temp = slot.Empty( );
timeData.AddTime(temp.GetAccumulatedTime( ));
if (resetTimeOnLeaving){
temp.ResetTimer( );
}
temp.IsFinished = true;
temp.ReturnToOrigin( );
}
}

Entity database 104 may be populated based on (1) random distribution, (2) historical data, and/or (3) based on real world input (e.g., real time parking lot occupancy data, sanitized medical profile data of preregistered patients, etc.). For example, a plurality of individuals may be generated and stored as entities having attributes (e.g., height, weight, etc.) selected randomly from bounded attribute value ranges. In other instances, the distribution of entity attribute values may be derived in some form from historical data from a previous simulation. In yet other instances, data may be updated in real-time or near real-time. For example, a user may operate the system at 3X normal speed to see a simulated future time for a vaccination site. The present state of the system derived from the entity database may be updated in real time, as may the utilization of slots at various junctions, from gathered data. For example, if data is received from the parking lot indicating a surge in the arrival of individuals, this surge may be represented in the number and distribution of individual entities flowing through the system.

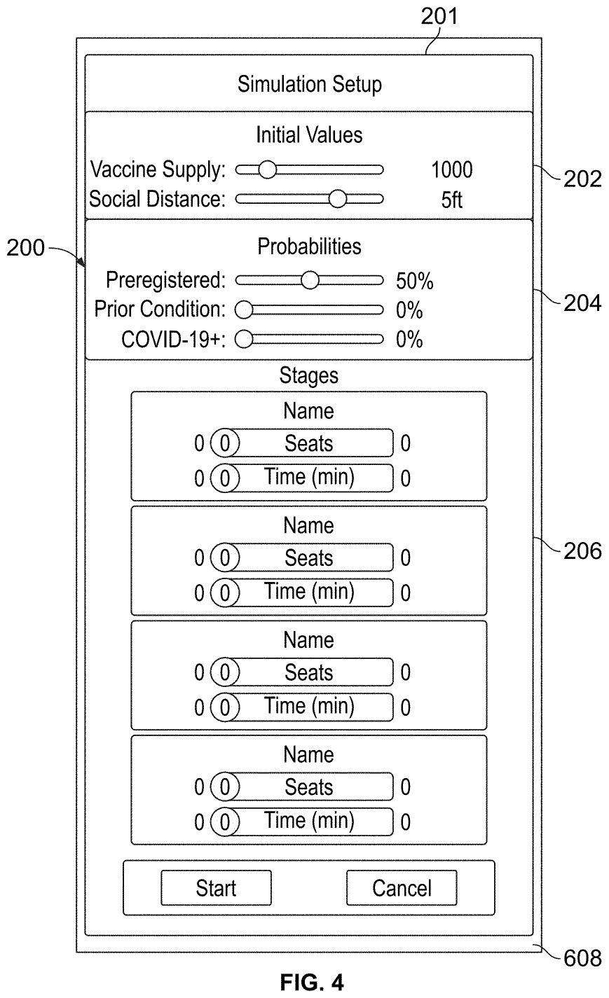

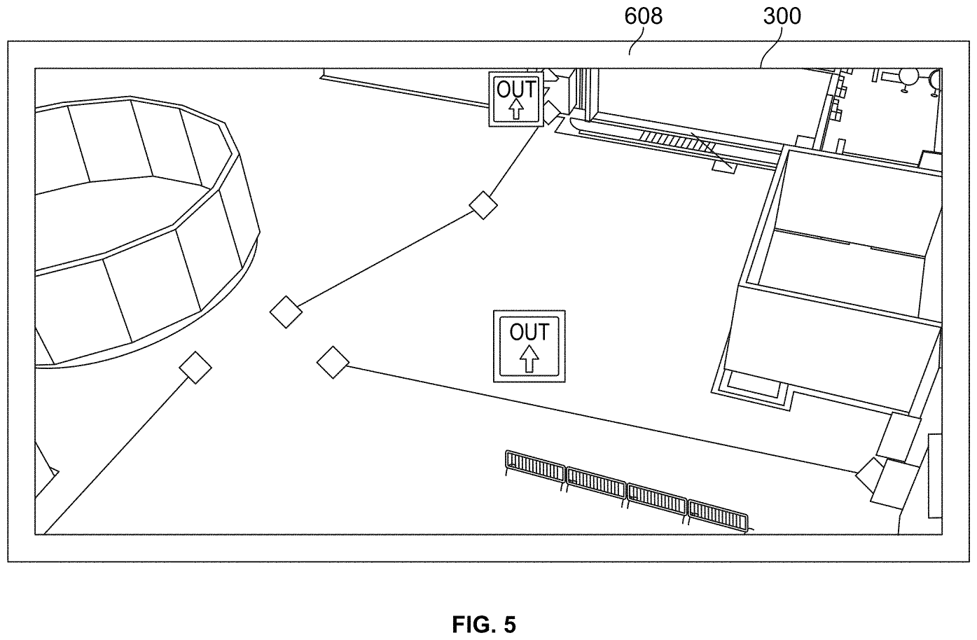

In accordance with exemplary and non-limiting embodiments, the parameters that define a simulation may be entered by a user via, for example, a graphical user interface (GUI) window such as window 200 displayed on a display device 608 of the computing system RBESDS 600, as shown in FIG. 4. With reference to FIG. 4, there is illustrated an exemplary and non-limiting embodiment of a simulation setup GUI window 200 element. For example, a user may press a Start button to begin simulation setup on the main GUI window screen displayed on the display device 608. In response, a menu 201 may be displayed within the window 200 with textual and/or graphical information displayed that prompts a user to provide input as for parameters to setup the simulation. As illustrated, the top panel 202 may include prompts to set the initial values, including the vaccine supply and social distancing between patients. The second panel 204 may display, for example, prompts to provide input setting probabilities including the number of arrivals who have preregistered, the probability an arrival has conditions that would require them to be observed for 30 minutes, and the probability that they have COVID-19 symptoms. The bottom panel 206 provides input prompts representing each stage at the simulated site. Adjusting the sliders may, for example, increase or decrease the maximum number of attendees at the stage. The more attendees, the more patients can be processed in parallel. The second slider is the expected amount of time it will take to complete the stage. This is used as a base time to accomplish a task. When ready, a user may press the Start button to setup the simulation with the selected values, and thereby cause display on the display device 608 of a simulated (e.g., virtual reality-type) environment (see, e.g., FIG. 5) in visual form that corresponds to the input provided and the rules/elements discussed above.

In accordance with some embodiments, after the RBESDS 600 displays a perspective view of the simulated environment, a user may use a mouse-type input device 606 to navigate the virtual environment and cause corresponding displays on the display device/output device 608. For example, left clicking the mouse on the displayed ground may move the camera to a location corresponding to the cursor while scrolling the mouse wheel may cause redisplay of the location as zoomed-in or zoomed-out.

In accordance with exemplary and non-limiting embodiments, various visual data indicators may be displayed by the RBESDS 600 on the display device 608 including, but not limited to, the number of patients currently on-site, the number of vaccines remaining on-site, the number of parking spaces currently occupied, the number of patients successfully vaccinated, and the rate of passage of time in the simulation.

Sources and sinks may add or remove entities from the simulation. Sources are indicated with a triangle facing downward with a plus sign. When connected to a database that provides entities, a green icon depicting three people is shown indicating it is properly configured. Sinks may be indicated with a triangle facing upward and a red minus sign. The RBESDS 600 provides this display via a GUI window of a display device 608.

In some embodiments, lines may be created through a game object with a line component attached. The path the line follows may be created using an empty game object named Path. Each child game object under path becomes the vertices of the line and their name does not matter, only their order. With reference to FIGS. 3A-3D, there is illustrated an exemplary and non-limiting embodiment of a perspective view of the simulated environment showing paths visualized as a series of grey dots connected by yellow lines. The RBESDS 600 provides this display via a GUI window of a display device 608.

In some embodiments, lines may be created via a game object with a Line component attached. The path the line follows may be created using an empty game object named Path. Each child game object under path becomes the vertices of the line and their name does not matter, only their order.

As described, a user interface may be utilized to adjust default values and to add base class attributes and associated functionality.

In addition to a logical system comprised of logical relationships and data flow rules, there exists a parallel simulated physical environment that may be mapped to the logical system. For example, as described above, entities may flow, logically, from a parking lot junction to a waiting area junction, e.g., according to the logic captured in the flow diagram of FIGS. 3A-3D. While the input/output nature of this definition has no time component, the spatial system operates to simulate the time needed for the patient to physically walk from the parking lot to the waiting area. In order to accurately present a displayed visualization of a physical person moving through physical space to accomplish such a task on a display device 608 of the RBESDS 600, there may exist a parallel representation and definition of the physical locations of the parking lot and waiting area. FIG. 5 illustrates an exemplary graphical user interface window 300 for display on a display device 608 by the RBESDS 600 to display a portion of a simulated environment with which a user may interact (using an input device 602) and observe the simulation modeling via the display device 608.

Working together, these two systems allow for the customization and execution of simulated tasks such as moving patients through a vaccination environment.

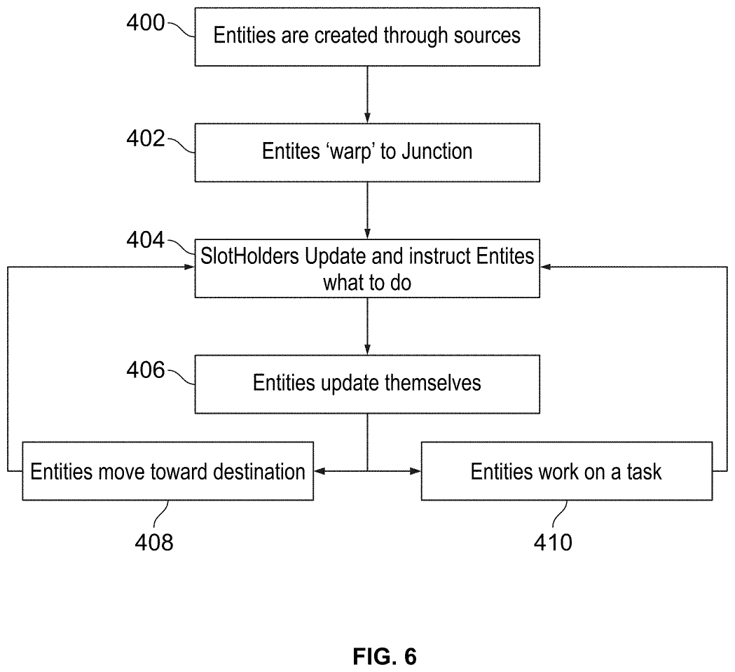

With reference to FIG. 6, there is illustrated an exemplary and non-limiting embodiment of a flowchart illustrating the manner in which entities are created, move and are updated. Specifically, at block 400, entities are created through sources. Sources may include external databases, may be statically stored and may be dynamically updated and/or created. At block 402, entities are warped to a junction. At block 404, slot holders updater and instruct each entity with regards to what they are to do. Entities proceed to update themselves at block 406 before moving towards the next destination at block 408 or working on a task at block 410. In either event, each entity proceeds to block 404 to be updated and instructed once again.

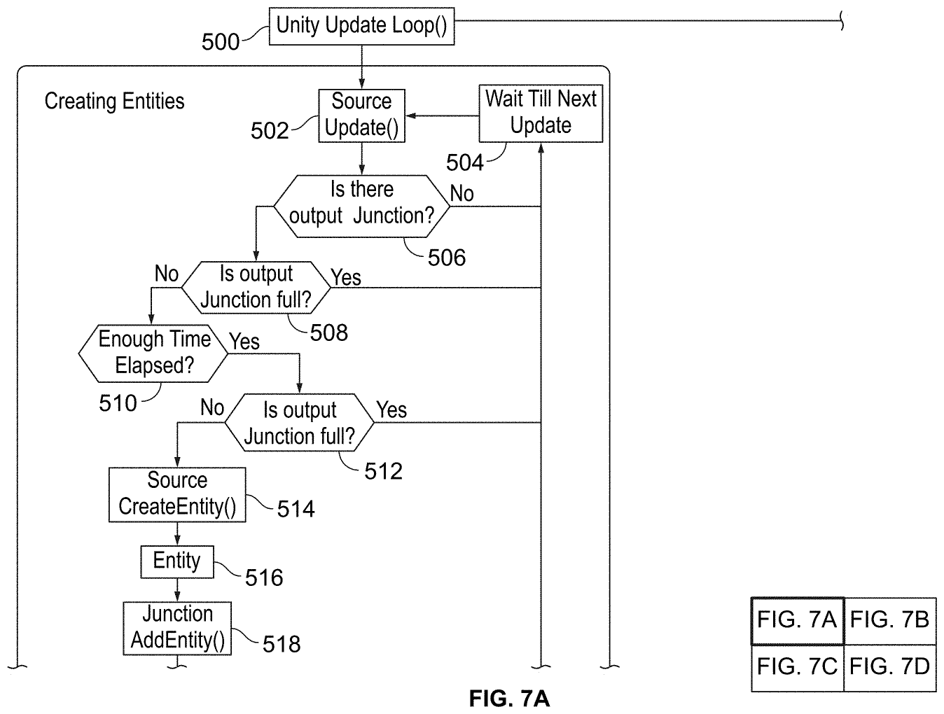

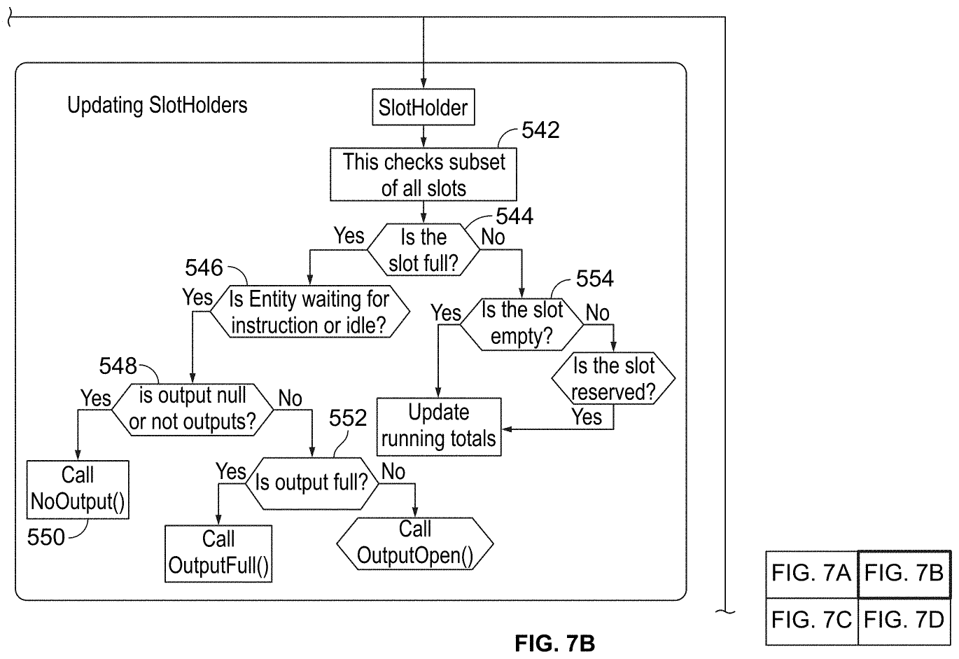

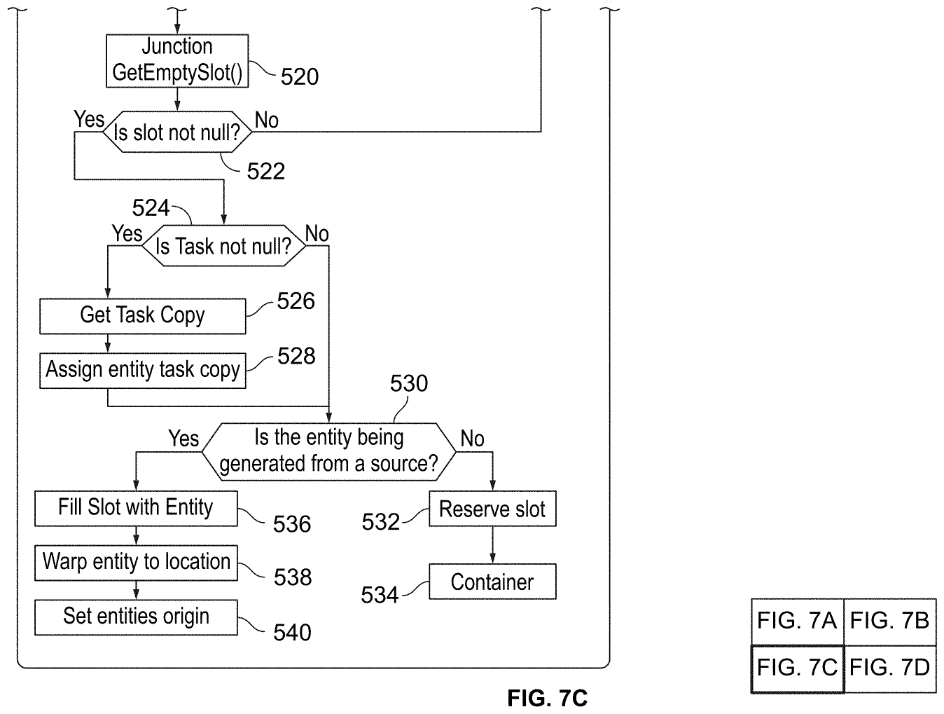

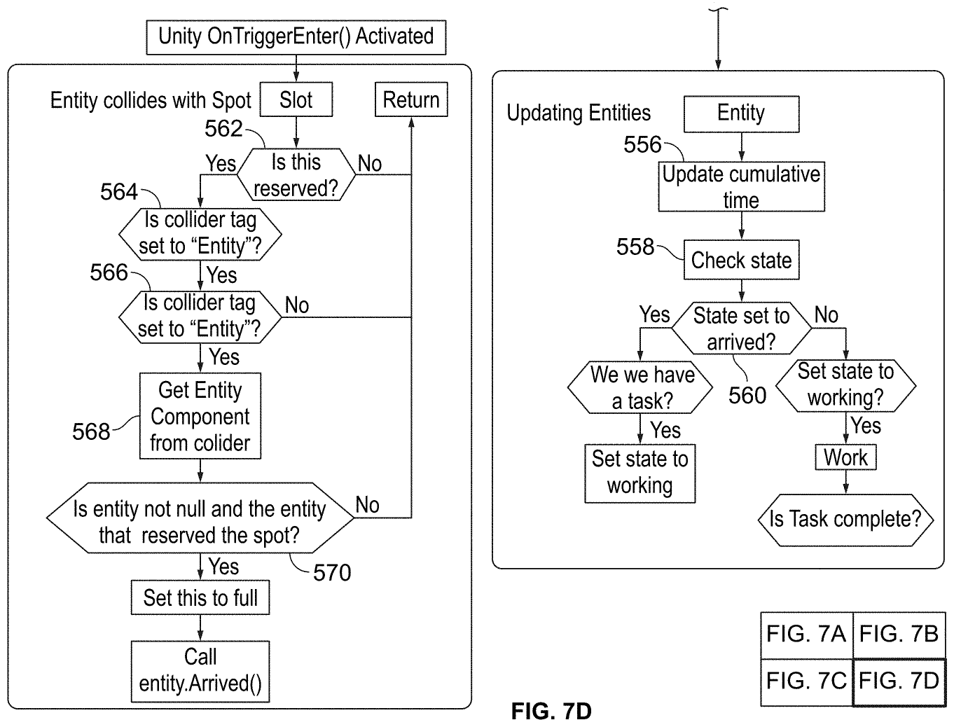

With reference to FIGS. 7A-7D, there is illustrated an exemplary and non-limiting embodiment of a flowchart for creating and updating entities and slot holders. As illustrated, there is executed an Update Loop command 500. Next, in series or in parallel, such as via the spawning of one or more threads, functionality to create an entity, update a slot holder and update an entity is performed.

To create an entity, a source update 502 is performed. Next, a check is performed at block 506 to determine if there is an output junction. If not, processing proceeds to block 504 whereat the system waits until the next update. If so, processing continues block 508 to determine if the output junction is full. If so, processing proceeds to block 504 whereat the system waits until the next update. If not, processing continues block 510 to determine if enough time has elapsed. If so, processing continues block 512 to determine if the output junction is full. If so, processing proceeds to block 504 whereat the system waits until the next update. If not, processing continues block 514 to invoke a create entity function, outputting entity 516 as the input to the add entity junction 518 which outputs to Get Empty Slot junction 520 and arriving at block 522 whereat there is determined if the slot is not full. If not, processing proceeds to block 504 whereat the system waits until the next update. If so, processing continues block 524 to determine if the task is not null. If not, processing proceeds to block 504 whereat the system waits until the next update. If not, processing continues block 530 to determine if the entity is being generated from a source. If so, task copy is received at block 526 and an entity task copy is assigned at block 528 before continuing to block 530.

At block 530, it is determined if the entity is being generated from a source. If not, a slot is reserved at block 532 and a container generated at block 534. If so, the slot is filled with an entity at block 536 the entity is warped to a location at block 538 and the entity's origin is set at block 540.

To update a slot holder, a subset of all slots is checked at block 542. At block 544, is check is performed to see if the slot is full. If so, a check is performed to see if the entity is waiting for an instruction or is idle at block 546. If so, a check is performed to see if the output is null at block 548. If so, a call is made indicate no output. If not, a check is performed to see if the output is full at block 552. If so, a call is made indicate output full. If not, a call is made indicate output open.

If, at block 544 it is determined that the slot is not full, processing continues to block to determine if the slot is empty. If so, running totals are updated. If not, is determination is made if the slot is reserved before updating running totals.

To update an entity, an entity serves as input to blocks 556, 558 and 560 whereat cumulative time is updated, a state is checked a state is checked to see if it is set to Arrived at block 560. If so, a determination of a task is made and, if so, the state is set to working. If the state is not set to Arrived, the state is set to working and a determination is made if the task is complete.

At runtime, the RBESDS 600 may display to a user a perspective rendering of an environment comprising the attributes of a logical system and a corresponding physical system FIG. 5 shows an exemplary graphical user interface window 300 for display on a display device 608 by the RBESDS 600 to display a portion of a simulated environment with which a user may interact (using an input device 602) and observe the simulation modeling via the display device 608.

Accordingly, the display device of the RBESDS 600 is controlled to display a graphical user interface providing a visual representation of a simulated environment that functions in a manner corresponding to data a database providing simulation information, which is composed of user defined rule-based junctions, spatial relationships, simulation attributes and entities, as discussed above. Initial setup of the simulator display provides all user-defined attributes that will be used to perform the simulation, generate the corresponding simulated environment, and provide the corresponding display via a graphical user interface window on the display device, as shown in the exemplary window 200 of FIG. 4. Each of the aforementioned elements, comprising data in the database for the environmental simulator display, are used to populate the 3D view displayed via the display device, such as the exemplary graphical user interface window 300 displayed in FIG. 5. Using the rules outlined by the user, an example of which can be seen in FIGS. 3A-3D, the RBESDS 600 system then updates, as shown in the flow diagram of FIGS. 7A-7D. Each entity referenced in the flow diagram, and used to control the display device to provide the simulation, follows the process shown in FIG. 6. Upon completion of an update, the RBESDS 600 displays the corresponding information to the user via a graphical display window displayed on a display device, providing spatial information of all junctions, entities, and attributes. The user can toggle what information is presented via the RBESDS 600 by providing input to a graphical interface. Results of the simulation can be stored locally or distributed through a communications network, as shown in FIG. 1. This sequence occurs until one or more user-defined conditions are met. Results of the simulation are then displayed to the user by the RBESDS 600, at which time the user may make changes to the rules, spatial representation, or attributes and initiate a new simulation.

In some embodiments, a user may define one or more alarm conditions. For example, a user may define an alarm condition when the number of slots available for the main entrance line is not sufficient and causes a backup in the waiting area junction. When, during a simulation, this condition is observed, an alarm may be presented to a user of the system. In some instances, the system may employ Artificial Intelligence (AI) to suggest possible remedial actions in such instances. In some instances, AI may analyze the results of numerous simulations and/or data describing real life scenarios in order to identify predictors of alarm conditions. In some instances, AI may operate to suggest adjustment to default or present data values in order to remedy identified alarm conditions.

In accordance with yet other exemplary and non-limiting embodiments, the system may provide a user interface for querying data generated during the course of a simulation. Such data may be presented in graphical form (e.g., a pie chart, a graph, a histogram, etc.) in order to enable manual analysis of a simulation.

As used throughout this application, the word “may” is used in a permissive sense (i.e., meaning having the potential to), rather than the mandatory sense (i.e., meaning must). The words “include,” “including,” and “includes” and the like mean including, but not limited to. As used herein, the singular form of “a,” “an,” and “the” include plural references unless the context clearly dictates otherwise. As employed herein, the term “number” shall mean one or an integer greater than one (i.e., a plurality).

As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other. Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

These drawings may not be drawn to scale and may not precisely reflect structure or performance characteristics of any given exemplary implementation, and should not be interpreted as defining or limiting the range of values or properties encompassed by exemplary implementations.

Unless specifically stated otherwise, as apparent from the discussion, it is appreciated that throughout this specification discussions utilizing terms such as “processing,” “computing,” “calculating,” “determining,” or the like refer to actions or processes of a specific apparatus, such as a special purpose computer or a similar special purpose electronic processing/computing system.

This disclosure includes a non-limiting set of embodiments used to describe certain inventions.

The various implementations and examples shown above illustrate a method and system for user interface management that provides a display of an environmental simulation according to predefined rules, using an electronic device. As is evident from the foregoing description, certain aspects of the present implementation are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present implementation. Accordingly, the specification and drawings are to be regarded in an illustrative rather than a restrictive sense.

Certain systems, apparatus, applications or processes are described herein as including a number of modules. A module may be a unit of distinct functionality that may be presented in software, hardware, or combinations thereof. When the functionality of a module is performed in any part through software, the module includes a computer-readable medium. The modules may be regarded as being communicatively coupled. The inventive subject matter may be represented in a variety of different implementations of which there are many possible permutations.

The methods described herein do not have to be executed in the order described, or in any particular order. Moreover, various activities described with respect to the methods identified herein can be executed in serial or parallel fashion. In the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

In an exemplary embodiment, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a server computer, a client computer, a personal computer (PC), a tablet PC, a set-top box (STB), a Personal Digital Assistant (PDA), a cellular telephone, a smart phone, a web appliance, a network router, switch or bridge, or any machine capable of executing a set of instructions (sequential or otherwise) that specify actions to be taken by that machine or computing device. Further, while only a single machine is illustrated, the term “machine” shall also be taken to include any collection of machines that individually or jointly execute a set (or multiple sets) of instructions to perform any one or more of the methodologies discussed herein.

The example computer system and client computers include a processor (e.g., a central processing unit (CPU), a graphics processing unit (GPU) or both), a main memory and a static memory, which communicate with each other via a bus. The computer system may further include a video/graphical display unit (e.g., a liquid crystal display (LCD) or a cathode ray tube (CRT)). The computer system and client computing devices also include an alphanumeric input device (e.g., a keyboard or touch-screen), a cursor control device (e.g., a mouse or gestures on a touch-screen), a drive unit, a signal generation device (e.g., a speaker and microphone) and a network interface device.

The system may include a computer-readable medium on which is stored one or more sets of instructions (e.g., software) embodying any one or more of the methodologies or systems described herein. The software may also reside, completely or at least partially, within the main memory and/or within the processor during execution thereof by the computer system, the main memory and the processor also constituting computer-readable media. The software may further be transmitted or received over a network via the network interface device.

The term “computer-readable medium” should be taken to include a single medium or multiple media (e.g., a centralized or distributed database, and/or associated caches and servers) that stores the one or more sets of instructions. The term “computer-readable medium” shall also be taken to include any medium that is capable of storing or encoding a set of instructions for execution by the machine and that cause the machine to perform any one or more of the methodologies of the present implementation. The term “computer-readable medium” shall accordingly be taken to include, but not be limited to, solid-state memories, and optical media, and magnetic media.

The present invention may be operational with numerous other general-purpose or special-purpose computing system environments or configurations. Examples of well-known computing systems, environments, and/or configurations that may be suitable for use with the present invention include, by way of example only, personal computers, server computers, hand-held or laptop devices, multiprocessor systems, microprocessor-based systems, set top boxes, programmable consumer electronics, cellular telephones, network PCs, minicomputers, mainframe computers, distributed computing environments that include any of the above-mentioned systems or devices, and the like.

The present invention has been described in the general context of computer-executable instructions, such as program modules or engines, being executed by a computer. Generally, program modules/engines include, but are not limited to, routines, programs, objects, components, and data structures that perform particular tasks or implement particular abstract data types. The present invention may also be practiced in distributed computing environments where tasks are performed by remote processing devices that are linked through a communications network. In a distributed computing environment, program modules/engines may be located in local and/or remote computer-storage media including, by way of example only, memory storage devices.

The exemplary computing system may include general-purpose computing hardware in the form of a server. Components of the server may include, without limitation, a processing unit, internal system memory, and a suitable system bus for coupling various system components, including a database cluster, with the server. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

The server typically includes therein, or has access to, a variety of computer-readable media, for instance, via a database cluster. Computer-readable media can be any available media that may be accessed by the server, and includes volatile and nonvolatile media, as well as removable and non-removable media. By way of example, and not limitation, computer-readable media may include computer-storage media and communication media. Computer-storage media may include, without limitation, volatile and nonvolatile media, as well as removable and non-removable media implemented in any method or technology for storage of information, such as computer readable instructions, data structures, program modules, or other data. In this regard, computer-storage media may include, but is not limited to, RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information, and which may be accessed by the server. Communication media typically embodies computer readable instructions, data structures, program modules, or other data in a modulated data signal, such as a carrier wave or other transport mechanism, and may include any information delivery media. The term “modulated data signal” refers to a signal that has one or more of its attributes set or changed in such a manner as to encode information in the signal. By way of example, and not limitation, communication media includes wired media such as a wired network or direct-wired connection, and wireless media such as acoustic, RF, infrared, and other wireless media. Combinations of any of the above also may be included within the scope of computer-readable media.

The server may operate in a computer network using logical connections to one or more remote computers. Remote computers may be located at a variety of locations or over the Internet. The remote computers may be personal computers, servers, routers, network PCs, peer devices, other common network nodes, or the like, and may include some or all of the elements described above in relation to the server. The computing devices can be personal digital assistants or other like devices.

Exemplary computer networks may include, without limitation, local area networks (LANs) and/or wide area networks (WANs). Such networking environments are commonplace in offices, enterprise-wide computer networks, intranets, and the Internet. When utilized in a WAN networking environment, the server may include a modem/network card or other means for establishing communications over the WAN, such as the Internet. In a networked environment, program modules or portions thereof may be stored in the server, in the database cluster, or on any of the remote computers. For example, and not by way of limitation, various application programs may reside on the memory associated with any one or more of the remote computers. It will be appreciated by those of ordinary skill in the art that the network connections shown are exemplary and other means of establishing a communications link between the computers (e.g., the server and remote computers) may be utilized.

In operation, a user may enter commands and information into the server or convey the commands and information to the server via one or more of the remote computers through input devices, such as a keyboard, a pointing device (commonly referred to as a mouse), a trackball, or a touch pad. Other input devices may include, without limitation, microphones, satellite dishes, scanners, or the like. Commands and information may also be sent directly from a remote device to the server. In addition to a monitor, the server and/or remote computers may include other peripheral output devices, such as speakers and a printer.

Many other internal components of the server and the remote computers/computing devices are not shown because such components and their interconnection are well known. Accordingly, additional details concerning the internal construction of the server and the remote computers/computing devices are not further disclosed herein.

Although methods and systems of embodiments of the present invention may be implemented in a WINDOWS or LINUX operating system, operating in conjunction with an Internet-based delivery system, one of ordinary skill in the art will recognize that the described methods and systems can be implemented in any system supporting the functionality described herein. As contemplated by the language above, the methods and systems of embodiments of the present invention may also be implemented on a stand-alone desktop, personal computer, cellular phone, smart phone, tablet, PDA, or any other computing device used in various locations.

Additionally, computer readable media storing computer readable code for carrying out the method steps identified above is provided. The computer readable media stores code for carrying out subprocesses for carrying out the methods described herein.

A computer program product recorded on a computer readable medium for carrying out the method steps identified herein is provided. The computer program product comprises computer readable means for carrying out the methods described above.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A computer product, comprising:
a logical environment system adapted to generate a logical network comprising a plurality of logically related, rule based junctions through which one or more entities traverse;
a spatial environment system adapted to map one or more spatial constraints upon each logical relation between each of the one or more of the plurality of junctions; and
a rendering environment system adapted to move the one or more entities through the logical network subject to the corresponding spatial constraints and to visually display the results of the movement of the one or more entities via a graphical user interface.

2. The computer product of claim 1, wherein at least one rule comprising a part of a rule based junction is a default rule.

3. The computer product of claim 1, wherein at least one rule comprising a part of a rule based junction is defined by a user of the computer product.

4. The computer product of claim 1, wherein a logical structure of each of the one or more entities is defined by a user of the computer product.

5. The computer product of claim 1, wherein at least one of the spatial is defined by a user of the computer product.

6. The computer product of claim 1, wherein each of the plurality of rule based junctions comprises an associated number of slots each adapted for holding a single entity.

7. The computer product of claim 6, wherein the number of slots associated with a single junction may be dynamically updated.

8. A method, comprising:
generating a logical network comprising a plurality of logically related, rule based junctions through which one or more entities traverse;
mapping one or more spatial constraints upon each logical relation between each of the one or more of the plurality of junctions; and
moving the one or more entities through the logical network subject to the corresponding spatial constraints and visually displaying the results of the movement of the one or more entities via a graphical user interface.

9. The method of claim 8, wherein at least one rule comprising a part of a rule based junction is a default rule.

10. The method of claim 8, wherein at least one rule comprising a part of a rule based junction is defined by a user of the computer product.

11. The method of claim 8, wherein a logical structure of each of the one or more entities is defined by a user of the computer product.

12. The method of claim 8, wherein at least one of the spatial is defined by a user of the computer product.

13. The method of claim 8, wherein each of the plurality of rule based junctions comprises an associated number of slots each adapted for holding a single entity.

14. The method of claim 13, wherein the number of slots associated with a single junction may be dynamically updated.

15. A system comprising:
a user input device;
a memory comprising a non-transitory data processor-readable medium;
a data processor operatively connected to said memory, said display device and said user input device; and
user interface management instructions embodied in data processor-executable code stored in the memory, said user interface management instructions being executable by the data processor to provide a user interface management engine configured to:
generate a logical network comprising a plurality of logically related, rule based junctions through which one or more entities traverse;
map one or more spatial constraints upon each logical relation between each of the one or more of the plurality of junctions; and
move the one or more entities through the logical network subject to the corresponding spatial constraints and visually displaying the results of the movement of the one or more entities via a graphical user interface.

16. The method of claim 15, wherein at least one rule comprising a part of a rule based junction is a default rule.

17. The method of claim 15, wherein at least one rule comprising a part of a rule based junction is defined by a user of the computer product.

18. The method of claim 15, wherein a logical structure of each of the one or more entities is defined by a user of the computer product.

19. The method of claim 15, wherein at least one of the spatial is defined by a user of the computer product.

20. The method of claim 15, wherein each of the plurality of rule based junctions comprises an associated number of slots each adapted for holding a single entity.

21. The method of claim 20, wherein the number of slots associated with a single junction may be dynamically updated.
